SEQUENCE LISTING (126 sequences)

SEQ ID NO: 1 - DNA, 19 bp, artificial sequence
  crook DNA
tcacctcatcccgcgaagc

SEQ ID NO: 2 - DNA, 14070 bp, homo sapiens
gaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcatcctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactgtccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagttctgaaagaatctcaacttccaactgtcatggacttcagaaaattctctcggaactatcaactctacaaatctgtttctattccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaacctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgatgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagactctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgccattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaacttaatgttttgggaacacacaaaatcgaagatggtacgttagcctctaagactaaaggaacatttgcacaccgtgacttcagtgcagaatatgaagaagatggcaaatatgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcaggaccatgctgagtgggtttatcaaggggccattagggaaattgatgatatcgacgagaggttccagaaaggagccagtgggaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcaatgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaaatgcctatattgatcaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcat

SEQ ID NO: 3 - DNA, 40 bp, artificial sequence
  ApoB crook
gucaucacacugaauaccaautcacctcatcccgcgaagc

SEQ ID NO: 4 - DNA, 21 bp, artificial sequence
  ApoB derived
auugguauucagugugaugac

SEQ ID NO: 5 - RNA, 21 nt, artificial sequence
  sense or antisense sequence
gagguguauggcuucaacccu

SEQ ID NO: 6 - RNA, 21 nt, artificial sequence
  sense or antisense sequence
agguguauggcuucaacccug

SEQ ID NO: 7 - RNA, 21 nt, artificial sequence
  sense or antisense sequence
gguguauggcuucaacccuga

SEQ ID NO: 8 - RNA, 21 nt, artificial sequence
  sense or antisense sequence
guguauggcuucaacccugag

SEQ ID NO: 9 - RNA, 21 nt, artificial sequence
  sense or antisense sequence
guauggcuucaacccugaggg

SEQ ID NO: 10 - RNA, 21 nt, artificial sequence
  sense or antisense sequence
auggcuucaacccugagggca

SEQ ID NO: 11 - RNA, 21 nt, artificial sequence
  sense or antisense sequence
uggcuucaacccugagggcaa

SEQ ID NO: 12 - RNA, 21 nt, artificial sequence
  sense or antisense sequence
cugaacaucaagaggggcauc

SEQ ID NO: 13 - RNA, 21 nt, artificial sequence
  sense or antisense sequence
ugaacaucaagaggggcauca

SEQ ID NO: 14 - RNA, 21 nt, artificial sequence
  sense or antisense sequence
gauaccguguauggaaacugc

SEQ ID NO: 15 - RNA, 21 nt, artificial sequence
  sense or antisense sequence
uaccguguauggaaacugcuc

SEQ ID NO: 16 - RNA, 21 nt, artificial sequence
  sense or antisense sequence
guccagccccaucacuuuaca

SEQ ID NO: 17 - RNA, 21 nt, artificial sequence
  sense or antisense sequence
cagccccaucacuuuacaagc

SEQ ID NO: 18 - RNA, 21 nt, artificial sequence
  sense or antisense sequence
agccccaucacuuuacaagcc

SEQ ID NO: 19 - RNA, 21 nt, artificial sequence
  sense or antisense sequence
gccccaucacuuuacaagccu

SEQ ID NO: 20 - RNA, 21 nt, artificial sequence
  sense or antisense sequence
cuuuacaagccuugguucagu

SEQ ID NO: 21 - RNA, 21 nt, artificial sequence
  sense or antisense sequence
acaagccuugguucagugugg

SEQ ID NO: 22 - RNA, 21 nt, artificial sequence
  sense or antisense sequence
aagccuugguucaguguggac

SEQ ID NO: 23 - RNA, 21 nt, artificial sequence
  sense or antisense sequence
ucacauccuccaguggcugaa

SEQ ID NO: 24 - RNA, 21 nt, artificial sequence
  sense or antisense sequence
aaauagaagggaaucuuauau

SEQ ID NO: 25 - RNA, 21 nt, artificial sequence
  sense or antisense sequence
uagaagggaaucuuauauuug

SEQ ID NO: 26 - RNA, 21 nt, artificial sequence
  sense or antisense sequence
gaagggaaucuuauauuugau

SEQ ID NO: 27 - RNA, 21 nt, artificial sequence
  sense or antisense sequence
ggaaucuuauauuugauccaa

SEQ ID NO: 28 - RNA, 21 nt, artificial sequence
  sense or antisense sequence
gaguuugugacaaauaugggc

SEQ ID NO: 29 - RNA, 21 nt, artificial sequence
  sense or antisense sequence
guuugugacaaauaugggcau

SEQ ID NO: 30 - RNA, 21 nt, artificial sequence
  sense or antisense sequence
gugacaaauaugggcaucauc

SEQ ID NO: 31 - RNA, 21 nt, artificial sequence
  sense or antisense sequence
agaugaacaccaacuucuucc

SEQ ID NO: 32 - RNA, 21 nt, artificial sequence
  sense or antisense sequence
gaugaacaccaacuucuucca

SEQ ID NO: 33 - RNA, 21 nt, artificial sequence
  sense or antisense sequence
ugaacaccaacuucuuccacg

SEQ ID NO: 34 - RNA, 21 nt, artificial sequence
  sense or antisense sequence
gaacaccaacuucuuccacga

SEQ ID NO: 35 - RNA, 21 nt, artificial sequence
  sense or antisense sequence
acaccaacuucuuccacgagu

SEQ ID NO: 36 - RNA, 21 nt, artificial sequence
  sense or antisense sequence
caccaacuucuuccacgaguc

SEQ ID NO: 37 - RNA, 21 nt, artificial sequence
  sense or antisense sequence
caaauggacucaucugcuaca

SEQ ID NO: 38 - RNA, 21 nt, artificial sequence
  sense or antisense sequence
ggacucaucugcuacagcuua

SEQ ID NO: 39 - RNA, 21 nt, artificial sequence
  sense or antisense sequence
ucugugggauuccaucugcca

SEQ ID NO: 40 - RNA, 21 nt, artificial sequence
  sense or antisense sequence
auuccaucugccaucucgaga

SEQ ID NO: 41 - RNA, 21 nt, artificial sequence
  sense or antisense sequence
acaauuugaucaguauauuaa

SEQ ID NO: 42 - RNA, 21 nt, artificial sequence
  sense or antisense sequence
caauuugaucaguauauuaaa

SEQ ID NO: 43 - RNA, 21 nt, artificial sequence
  sense or antisense sequence
uaaaucaagugucaucacacu

SEQ ID NO: 44 - RNA, 21 nt, artificial sequence
  sense or antisense sequence
aucaagugucaucacacugaa

SEQ ID NO: 45 - RNA, 21 nt, artificial sequence
  sense or antisense sequence
ucaagugucaucacacugaau

SEQ ID NO: 46 - RNA, 21 nt, artificial sequence
  sense or antisense sequence
caagugucaucacacugaauu

SEQ ID NO: 47 - RNA, 21 nt, artificial sequence
  sense or antisense sequence
gucaucacacugaauaccaau

SEQ ID NO: 48 - RNA, 21 nt, artificial sequence
  sense or antisense sequence
ucaucacacugaauaccaaug

SEQ ID NO: 49 - RNA, 21 nt, artificial sequence
  sense or antisense sequence
caucacacugaauaccaaugc

SEQ ID NO: 50 - RNA, 21 nt, artificial sequence
  sense or antisense sequence
uaacacuaagaaccagaagau

SEQ ID NO: 51 - RNA, 21 nt, artificial sequence
  sense or antisense sequence
auugggaagaagaggcagcuu

SEQ ID NO: 52 - RNA, 21 nt, artificial sequence
  sense or antisense sequence
gauugauugaccuguccauuc

SEQ ID NO: 53 - RNA, 21 nt, artificial sequence
  sense or antisense sequence
ugauugaccuguccauucaaa

SEQ ID NO: 54 - RNA, 21 nt, artificial sequence
  sense or antisense sequence
gauugaccuguccauucaaaa

SEQ ID NO: 55 - RNA, 21 nt, artificial sequence
  sense or antisense sequence
gaccuguccauucaaaacuac

SEQ ID NO: 56 - RNA, 21 nt, artificial sequence
  sense or antisense sequence
accuguccauucaaaacuacc

SEQ ID NO: 57 - RNA, 21 nt, artificial sequence
  sense or antisense sequence
cuguccauucaaaacuaccac

SEQ ID NO: 58 - RNA, 21 nt, artificial sequence
  sense or antisense sequence
aggguugaagccauacaccuc

SEQ ID NO: 59 - RNA, 21 nt, artificial sequence
  sense or antisense sequence
caggguugaagccauacaccu

SEQ ID NO: 60 - RNA, 21 nt, artificial sequence
  sense or antisense sequence
ucaggguugaagccauacacc

SEQ ID NO: 61 - RNA, 21 nt, artificial sequence
  sense or antisense sequence
cucaggguugaagccauacac

SEQ ID NO: 62 - RNA, 21 nt, artificial sequence
  sense or antisense sequence
cccucaggguugaagccauac

SEQ ID NO: 63 - RNA, 21 nt, artificial sequence
  sense or antisense sequence
ugcccucaggguugaagccau

SEQ ID NO: 64 - RNA, 21 nt, artificial sequence
  sense or antisense sequence
uugcccucaggguugaagcca

SEQ ID NO: 65 - RNA, 21 nt, artificial sequence
  sense or antisense sequence
gaugccccucuugauguucag

SEQ ID NO: 66 - RNA, 21 nt, artificial sequence
  sense or antisense sequence
ugaugccccucuugauguuca

SEQ ID NO: 67 - RNA, 21 nt, artificial sequence
  sense or antisense sequence
gcaguuuccauacacgguauc

SEQ ID NO: 68 - RNA, 21 nt, artificial sequence
  sense or antisense sequence
gagcaguuuccauacacggua

SEQ ID NO: 69 - RNA, 21 nt, artificial sequence
  sense or antisense sequence
uguaaagugauggggcuggac

SEQ ID NO: 70 - RNA, 21 nt, artificial sequence
  sense or antisense sequence
gcuuguaaagugauggggcug

SEQ ID NO: 71 - RNA, 21 nt, artificial sequence
  sense or antisense sequence
ggcuuguaaagugauggggcu

SEQ ID NO: 72 - RNA, 21 nt, artificial sequence
  sense or antisense sequence
aggcuuguaaagugauggggc

SEQ ID NO: 73 - RNA, 21 nt, artificial sequence
  sense or antisense sequence
acugaaccaaggcuuguaaag

SEQ ID NO: 74 - RNA, 21 nt, artificial sequence
  sense or antisense sequence
ccacacugaaccaaggcuugu

SEQ ID NO: 75 - RNA, 21 nt, artificial sequence
  sense or antisense sequence
guccacacugaaccaaggcuu

SEQ ID NO: 76 - RNA, 21 nt, artificial sequence
  sense or antisense sequence
uucagccacuggaggauguga

SEQ ID NO: 77 - RNA, 21 nt, artificial sequence
  sense or antisense sequence
auauaagauucccuucuauuu

SEQ ID NO: 78 - RNA, 21 nt, artificial sequence
  sense or antisense sequence
caaauauaagauucccuucua

SEQ ID NO: 79 - RNA, 21 nt, artificial sequence
  sense or antisense sequence
aucaaauauaagauucccuuc

SEQ ID NO: 80 - RNA, 21 nt, artificial sequence
  sense or antisense sequence
uuggaucaaauauaagauucc

SEQ ID NO: 81 - RNA, 21 nt, artificial sequence
  sense or antisense sequence
gcccauauuugucacaaacuc

SEQ ID NO: 82 - RNA, 21 nt, artificial sequence
  sense or antisense sequence
augcccauauuugucacaaac

SEQ ID NO: 83 - RNA, 21 nt, artificial sequence
  sense or antisense sequence
gaugaugcccauauuugucac

SEQ ID NO: 84 - RNA, 21 nt, artificial sequence
  sense or antisense sequence
ggaagaaguugguguucaucu

SEQ ID NO: 85 - RNA, 21 nt, artificial sequence
  sense or antisense sequence
uggaagaaguugguguucauc

SEQ ID NO: 86 - RNA, 21 nt, artificial sequence
  sense or antisense sequence
cguggaagaaguugguguuca

SEQ ID NO: 87 - RNA, 21 nt, artificial sequence
  sense or antisense sequence
ucguggaagaaguugguguuc

SEQ ID NO: 88 - RNA, 21 nt, artificial sequence
  sense or antisense sequence
acucguggaagaaguuggugu

SEQ ID NO: 89 - RNA, 21 nt, artificial sequence
  sense or antisense sequence
gacucguggaagaaguuggug

SEQ ID NO: 90 - RNA, 21 nt, artificial sequence
  sense or antisense sequence
uguagcagaugaguccauuug

SEQ ID NO: 91 - RNA, 21 nt, artificial sequence
  sense or antisense sequence
uaagcuguagcagaugagucc

SEQ ID NO: 92 - RNA, 21 nt, artificial sequence
  sense or antisense sequence
uggcagauggaaucccacaga

SEQ ID NO: 93 - RNA, 21 nt, artificial sequence
  sense or antisense sequence
ucucgagauggcagauggaau

SEQ ID NO: 94 - RNA, 21 nt, artificial sequence
  sense or antisense sequence
uuaauauacugaucaaauugu

SEQ ID NO: 95 - RNA, 21 nt, artificial sequence
  sense or antisense sequence
uuuaauauacugaucaaauug

SEQ ID NO: 96 - RNA, 21 nt, artificial sequence
  sense or antisense sequence
agugugaugacacuugauuua

SEQ ID NO: 97 - RNA, 21 nt, artificial sequence
  sense or antisense sequence
uucagugugaugacacuugau

SEQ ID NO: 98 - RNA, 21 nt, artificial sequence
  sense or antisense sequence
auucagugugaugacacuuga

SEQ ID NO: 99 - RNA, 21 nt, artificial sequence
  sense or antisense sequence
aauucagugugaugacacuug

SEQ ID NO: 100 - RNA, 21 nt, artificial sequence
  sense or antisense sequence
auugguauucagugugaugac

SEQ ID NO: 101 - RNA, 21 nt, artificial sequence
  sense or antisense sequence
cauugguauucagugugauga

SEQ ID NO: 102 - RNA, 21 nt, artificial sequence
  sense or antisense sequence
gcauugguauucagugugaug

SEQ ID NO: 103 - RNA, 21 nt, artificial sequence
  sense or antisense sequence
aucuucugguucuuaguguua

SEQ ID NO: 104 - RNA, 21 nt, artificial sequence
  sense or antisense sequence
aagcugccucuucuucccaau

SEQ ID NO: 105 - RNA, 21 nt, artificial sequence
  sense or antisense sequence
gaauggacaggucaaucaauc

SEQ ID NO: 106 - RNA, 21 nt, artificial sequence
  sense or antisense sequence
uuugaauggacaggucaauca

SEQ ID NO: 107 - RNA, 21 nt, artificial sequence
  sense or antisense sequence
uuuugaauggacaggucaauc

SEQ ID NO: 108 - RNA, 21 nt, artificial sequence
  sense or antisense sequence
guaguuuugaauggacagguc

SEQ ID NO: 109 - RNA, 21 nt, artificial sequence
  sense or antisense sequence
gguaguuuugaauggacaggu

SEQ ID NO: 110 - RNA, 21 nt, artificial sequence
  sense or antisense sequence
gugguaguuuugaauggacag

SEQ ID NO: 111 - RNA, 21 nt, artificial sequence
  sense or antisense sequence
cagcaccuagcuggaaaguua

SEQ ID NO: 112 - RNA, 21 nt, artificial sequence
  sense or antisense sequence
uaacuuuccagcuaggugcug

SEQ ID NO: 113 - RNA, 21 nt, artificial sequence
  sense or antisense sequence
cuccauggaauuuaaguauga

SEQ ID NO: 114 - RNA, 21 nt, artificial sequence
  sense or antisense sequence
ucauacuuaaauuccauggag

SEQ ID NO: 115 - RNA, 21 nt, artificial sequence
  sense or antisense sequence
uucccugaaguugauguguua

SEQ ID NO: 116 - RNA, 21 nt, artificial sequence
  sense or antisense sequence
uaacacaucaacuucagggaa

SEQ ID NO: 117 - RNA, 21 nt, artificial sequence
  sense or antisense sequence
guccaauaagaucaauagcaa

SEQ ID NO: 118 - RNA, 21 nt, artificial sequence
  sense or antisense sequence
uugcuauugaucuuauuggac

SEQ ID NO: 119 - RNA, 21 nt, artificial sequence
  sense or antisense sequence
aacucucaaacccuaagauua

SEQ ID NO: 120 - RNA, 21 nt, artificial sequence
  sense or antisense sequence
uaaucuuaggguuugagaguu

SEQ ID NO: 121 - RNA, 21 nt, artificial sequence
  sense or antisense sequence
ucggaacaauccucagaguua

SEQ ID NO: 122 - RNA, 21 nt, artificial sequence
  sense or antisense sequence
uaacucugaggauuguuccga

SEQ ID NO: 123 - RNA, 21 nt, artificial sequence
  sense or antisense sequence
aagcaagaacuuaauggaaau

SEQ ID NO: 124 - RNA, 21 nt, artificial sequence
  sense or antisense sequence
auuuccauuaaguucuugcuu

SEQ ID NO: 125 - RNA, 21 nt, artificial sequence
  sense or antisense sequence
ggccauuaggcaaauugauga

SEQ ID NO: 126 - RNA, 21 nt, artificial sequence
  sense or antisense sequence
ucaucaauuugccuaauggcc

(19) United States
(12) Patent Application Publication
(10) Pub. No.: US 2023/0027604 A1
(43) Pub. Date: Jan. 26, 2023

(54) APOLIPOPROTEIN B ANTAGONIST

(71) Applicant: Argonaute RNA Limited, Bristol (GB)

(72) Inventors: Michael Khan, Bristol (GB); Daniel Mitchell, Bristol (GB)

(73) Assignee: Argonaute RNA Limited, Bristol (GB)

(21) Appl. No.: 17/620,973

(22) PCT Filed: Jun. 30, 2020
(86) PCT No.: PCT/GB2020/051573

§ 371 Date: Dec. 20, 2021

(30) Foreign Application Priority Data
Jul. 2, 2019 (GB) 1909500.9
Jul. 23, 2019 (GB) 1910526.1
Jan. 22, 2020 (GB) 2000906.4

(51) Int. Cl. C12N 15/113; A61K 31/713; A61P 3/06

(52) U.S. Cl. CPC C12N 15/113; A61K 31/713; A61P 3/06; C12N 2310/14; C12N 2310/351; C12N 2310/3519; C12N 2320/11; C12N 2330/31

(57) ABSTRACT

This disclosure relates to a nucleic acid comprising a double stranded RNA molecule comprising sense and antisense strands and further comprising a single stranded DNA molecule covalently linked to the 3′ end of either the sense or antisense RNA part of the molecule wherein the double stranded inhibitory RNA targets apolipoprotein B in the treatment hypercholesterolemia.

### CROSS REFERENCE TO RELATED APPLICATIONS

This is the U.S. National Stage of International Application No. PCT/GB2020/051573, filed Jun. 30, 2020, which was published in English under PCT Article 21(2), which in turn claims the benefit of Great Britain Application No. 2000906.4, filed Jan. 22, 2020, Great Britain Application No. 1910526.1, filed Jul. 23, 2019, and Great Britain Application No. 1909500.9, filed Jul. 2, 2019.

### Incorporation of Electronic Sequence Listing

The Sequence Listing is submitted as an ASCII text file, created on Dec. 14, 2021, 42.7 KB, which is incorporated by reference herein.

### FIELD OF THE DISCLOSURE

This disclosure relates to a nucleic acid comprising a double stranded RNA molecule comprising sense and antisense strands and further comprising a single stranded DNA molecule covalently linked to the 3′ end of either the sense or antisense RNA part of the molecule wherein the double stranded inhibitory RNA targets apolipoprotein B (ApoB); pharmaceutical compositions comprising said nucleic acid molecule and methods for the treatment of diseases associated with increased levels of ApoB, for example hypercholesterolemia.

### BACKGROUND TO THE DISCLOSURE

Cardiovascular disease associated with hypercholesterolemia is a common condition and results in heart disease and a high incidence of death and morbidity and can be a consequence of poor diet, obesity or an inherited dysfunctional gene. For example, mutations in Low Density Lipoprotein Receptor (LDL-receptor) or apolipoprotein B (ApoB) as in familial hypercholesterolemia. Cholesterol is essential for membrane biogenesis in animal cells. The lack of water solubility means that cholesterol is transported around the body in association with lipoproteins. Apolipoproteins form together with phospholipids, cholesterol and lipids which facilitate the transport of lipids such as cholesterol, through the bloodstream to the different parts of the body. Lipoproteins are classified according to size and can form HDL (High-density lipoprotein), LDL (Low-density lipoprotein), IDL (intermediate-density lipoprotein), VLDL (very low-density lipoprotein) and ULDL (ultra-low-density lipoprotein) lipoproteins.

Lipoproteins change composition throughout their circulation comprising different ratios of apolipoproteins A (ApoA), B (ApoB), C (ApoC), D(ApoD) or E (ApoE), triglycerides, cholesterol and phospholipids. ApoB is the main apolipoprotein of ULDL and LDL and has two isoforms apoB-48 and apoB-100. Both ApoB isoforms are encoded by one single gene and wherein the shorter ApoB-48 gene is produced after RNA editing of the ApoB-100 transcript at residue 2180 resulting in the creation of a stop codon. ApoB-100 is the main structural protein of LDL and serves as a ligand for a cell receptor which allows transport of, for example, cholesterol into a cell.

Familial hypercholesterolemia is an orphan disease and results from elevated levels of LDL cholesterol (LDL-C) in the blood. The disease is an autosomal dominant disorder with both the heterozygous (350-550 mg/dL LDL-C) and homozygous (650-1000 mg/dL LDL-C) states resulting in elevated LDL-C. The heterozygous form of familial hypercholesterolemia is around 1:500 of the population. The homozygous state is much rarer and is approximately 1:1,000,000. The normal levels of LDL-C are in the region 130 mg/dL.

Hypercholesterolemia is particularly acute in paediatric patients which if not diagnosed early can result in accelerated coronary heart disease and premature death. If diagnosed and treated early the child can have a normal life expectancy. In adults, high LDL-C, either because of mutation or other factors, is directly associated with increased risk of atherosclerosis which can lead to coronary artery disease, stroke or kidney problems. Lowering levels of LDL-C is known to reduce the risk of atherosclerosis and associated conditions. LDL-C levels can be lowered initially by administration of statins which block the de novo synthesis of cholesterol by inhibiting the HMG-CoA reductase. Some subjects can benefit from combination therapy which combines a statin with other therapeutic agents such as ezetimibe, colestipol or nicotinic acid. However, expression and synthesis of HMG-CoA reductase adapts in response to the statin inhibition and increases over time, thus the beneficial effects are only temporary or limited after statin resistance is established.

There is therefore a desire to identify alternative therapies that can be used alone or in combination with existing therapeutic approaches to control cardiovascular disease because of elevated LDL-C.

A technique to specifically ablate gene function is through the introduction of double stranded inhibitory RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically, but not exclusively, derived from exons of the gene which is to be ablated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

ApoB is a known target for therapeutic intervention in the regulation of LDL-C. For example, attempts to silence ApoB synthesis by using antisense RNA is known in the art; see WO2006/053430, WO2008/109357, WO2014/076196, WO2010/076248, WO2015/071388, WO2011/000108 and WO2008/118883. A problem with administering RNAi or antisense oligonucleotides is the toxicity caused by modified, non-naturally occurring nucleotides or the length of the RNAi molecules. Moreover, because antisense techniques do not necessarily produce stable transformation the stability of the antisense constructs such as RNAi is variable.

This disclosure relates to a nucleic acid molecule comprising a double stranded inhibitory RNA that is modified by the inclusion of a short DNA part linked to the 3′ end of either the sense or antisense inhibitory RNA and which forms a hairpin structure and is designed with reference to the nucleotide sequence encoding ApoB. U.S. Pat. No. 8,067,572, which is incorporated by reference in its entirety, discloses examples of said nucleic acid molecules. The double stranded inhibitory RNA uses solely or predominantly natural nucleotides and does not require modified nucleotides or sugars that prior art double stranded RNA molecules typically utilise to improve pharmacodynamics and pharmacokinetics. The disclosed double stranded inhibitory RNAs have activity in silencing ApoB with potentially fewer side effects.

### STATEMENTS OF THE INVENTION

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human apolipoprotein B protein and wherein said single stranded DNA molecule comprises a nucleotide sequence that is adapted over at least part of its length to anneal by complementary base pairing to a part of said single stranded DNA to form a double stranded DNA structure.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human apolipoprotein B protein, or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure.

A “polymorphic sequence variant” is a sequence that varies by one, two, three or more nucleotides. Apo B polymorphisms are known in the art, some of which are associated with hypercholesterolemia.

In a preferred embodiment of the invention wherein the 5′ end of said single stranded DNA molecule is covalently linked to the 3′ end of the sense strand of the double stranded inhibitory RNA molecule.

In a preferred embodiment of the invention wherein the 5′ end of said single stranded DNA molecule is covalently linked to the 3′ end of the antisense strand of the double stranded inhibitory RNA molecule.

In a preferred embodiment of the invention said single stranded DNA molecule comprises the nucleotide sequence TCACCTCATCCCGCGAAGC (SEQ ID NO: 1).

In a preferred embodiment of the invention said double stranded inhibitory RNA molecule is between 10 and 40 nucleotides in length.

In a preferred embodiment of the invention said double stranded inhibitory RNA molecule is between 18 and 29 base pairs in length.

In a preferred embodiment of the invention said double stranded inhibitory RNA molecule is 21 base pairs in length.

In a preferred embodiment of the invention said double stranded inhibitory RNA is designed with reference to a nucleotide sequence as set forth in SEQ ID NO: 2.

In a preferred embodiment of the invention said double stranded inhibitory RNA molecule comprises a nucleotide sequence as set forth in SEQ ID NO: 3.

In a preferred embodiment of the invention said a double stranded inhibitory RNA molecule comprises a nucleotide sequence as set forth in SEQ ID NO: 4.

In an alternative embodiment of the invention said double stranded inhibitory RNA molecule comprises a sense nucleotide sequence selected from the group consisting of: SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57.

In an alternative embodiment of the invention said double stranded inhibitory RNA molecule comprises an antisense nucleotide sequence selected from the group consisting of: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 98, 99, 100, 101, 102, 103, 104, 105, 106, 107, 108, 109 and 110.

In a preferred embodiment of the invention said double stranded inhibitory RNA molecule comprises a sense nucleotide sequence set forth in SEQ ID NO: 27 and an antisense nucleotide sequence set forth in SEQ ID NO: 47.

In an alternative preferred embodiment of the invention said double stranded inhibitory RNA molecule comprises a sense nucleotide sequence set forth in SEQ ID NO: 80 and an antisense nucleotide sequence set forth in SEQ ID NO:100.

In an alternative embodiment of the invention said double stranded inhibitory RNA molecule comprises a sense nucleotide sequence selected from the group consisting of: 111, 113, 115, 117, 119, 121, 123 and 125.

In an alternative embodiment of the invention said double stranded inhibitory RNA molecule comprises a sense nucleotide sequence selected from the group consisting of: 112, 114, 116, 118, 120, 122, 124 and 126.

In a preferred embodiment of the invention said double stranded inhibitory RNA molecule comprises a sense nucleotide sequence set forth in SEQ ID NO: 111 and an antisense nucleotide sequence set forth in SEQ ID NO:112.

In a preferred embodiment of the invention said double stranded inhibitory RNA molecule comprises a sense nucleotide sequence set forth in SEQ ID NO: 113 and an antisense nucleotide sequence set forth in SEQ ID NO:114.

In a preferred embodiment of the invention said double stranded inhibitory RNA molecule comprises a sense nucleotide sequence set forth in SEQ ID NO: 115 and an antisense nucleotide sequence set forth in SEQ ID NO:116.

In a preferred embodiment of the invention said double stranded inhibitory RNA molecule comprises a sense nucleotide sequence set forth in SEQ ID NO: 117 and an antisense nucleotide sequence set forth in SEQ ID NO:118.

In a preferred embodiment of the invention said double stranded inhibitory RNA molecule comprises a sense nucleotide sequence set forth in SEQ ID NO: 119 and an antisense nucleotide sequence set forth in SEQ ID NO:120.

In a preferred embodiment of the invention said double stranded inhibitory RNA molecule comprises a sense nucleotide sequence set forth in SEQ ID NO: 121 and an antisense nucleotide sequence set forth in SEQ ID NO:122.

In a preferred embodiment of the invention said double stranded inhibitory RNA molecule comprises a sense nucleotide sequence set forth in SEQ ID NO: 123 and an antisense nucleotide sequence set forth in SEQ ID NO:124.

In a preferred embodiment of the invention said double stranded inhibitory RNA molecule comprises a sense nucleotide sequence set forth in SEQ ID NO: 125 and an antisense nucleotide sequence set forth in SEQ ID NO:126.

In an alternative embodiment of the invention said double stranded inhibitory RNA molecule comprises a sense nucleotide sequence selected from the group consisting of: SEQ ID NO: 7, SEQ ID NO: 36, SEQ ID NO: 111, SEQ ID NO: 113, SEQ ID NO: 115 and SEQ ID NO: 119.

In a preferred embodiment of the invention said double stranded inhibitory RNA molecule comprises an antisense nucleotide sequence selected from the group consisting of: SEQ ID NO: 60, SEQ ID NO: 72, SEQ ID NO: 89, SEQ ID NO: 100, SEQ ID NO: 108, SEQ ID NO: 114 and SEQ ID NO: 118.

In a preferred embodiment of the invention said double stranded inhibitory RNA molecule comprises a sense nucleotide sequence set forth in SEQ ID NO: 7 and an antisense nucleotide sequence set forth in SEQ ID NO:60.

In a preferred embodiment of the invention said double stranded inhibitory RNA molecule comprises a sense nucleotide sequence set forth in SEQ ID NO: 111 and an antisense nucleotide sequence set forth in SEQ ID NO:112.

In a preferred embodiment of the invention said double stranded inhibitory RNA molecule comprises a sense nucleotide sequence set forth in SEQ ID NO: 117 and an antisense nucleotide sequence set forth in SEQ ID NO:118.

In a preferred embodiment of the invention said double stranded inhibitory RNA molecule comprises a sense nucleotide sequence set forth in SEQ ID NO: 55 and an antisense nucleotide sequence set forth in SEQ ID NO:108.

In a preferred embodiment of the invention said double stranded inhibitory RNA molecule comprises a sense nucleotide sequence set forth in SEQ ID NO: 47 and an antisense nucleotide sequence set forth in SEQ ID NO:100.

In a preferred embodiment of the invention said double stranded inhibitory RNA molecule comprises a sense nucleotide sequence set forth in SEQ ID NO: 36 and an antisense nucleotide sequence set forth in SEQ ID NO:89.

In a preferred embodiment of the invention said double stranded inhibitory RNA molecule comprises a sense nucleotide sequence set forth in SEQ ID NO: 19 and an antisense nucleotide sequence set forth in SEQ ID NO:72.

In a preferred embodiment of the invention said double stranded inhibitory RNA molecule comprises a sense nucleotide sequence set forth in SEQ ID NO: 115 and an antisense nucleotide sequence set forth in SEQ ID NO:116.

In a preferred embodiment of the invention said double stranded inhibitory RNA molecule comprises a sense nucleotide sequence set forth in SEQ ID NO: 113 and an antisense nucleotide sequence set forth in SEQ ID NO: 114.

In a preferred embodiment of the invention said double stranded inhibitory RNA molecule comprises a sense nucleotide sequence set forth in SEQ ID NO: 119 and an antisense nucleotide sequence set forth in SEQ ID NO: 120.

In a preferred embodiment of the invention said double stranded inhibitory RNA molecule comprises a sense nucleotide sequence set forth in SEQ ID NO: 113 and an antisense nucleotide sequence set forth in SEQ ID NO:114.

In a preferred embodiment of the invention said double stranded inhibitory RNA molecule comprises a modified base, sugar, inter-nucleotide linkage, or combinations thereof.

In a preferred embodiment of the invention said nucleic acid molecule is covalently linked to a carrier molecule adapted to deliver said nucleic acid molecule to a cell or tissue.

In a preferred embodiment of the invention said nucleic acid molecule is covalently linked to N-acetylgalactosamine, Preferably, N-acetylgalactosamine is triantennary.

In an alternative preferred embodiment of the invention said nucleic acid molecule is covalently linked to oligornannose, oligofucose, or N-acetylgalactosamine 4-sulfate.

According to a further aspect of the invention there is provided a pharmaceutical composition comprising at least one nucleic acid molecule according to the invention.

In a preferred embodiment of the invention said composition further includes a pharmaceutical carrier and/or excipient.

When administered the compositions of the present invention are administered in pharmaceutically acceptable preparations. Such preparations may routinely contain pharmaceutically acceptable concentrations of salt, buffering agents, preservatives, compatible carriers and optionally other therapeutic agents, such as cholesterol lowering agents, which can be administered separately from the nucleic acid molecule according to the invention or in a combined preparation if a combination is compatible.

The combination of a nucleic acid according to the invention and the other, different therapeutic agent is administered as simultaneous, sequential or temporally separate dosages.

The therapeutics of the invention can be administered by any conventional route, including injection or by gradual infusion over time. The administration may, for example, be oral, intravenous, intraperitoneal, intramuscular, intracavity, subcutaneous, transdermal or transepithelial.

The compositions of the invention are administered in effective amounts. An “effective amount” is that amount of a composition that alone, or together with further doses, produces the desired response. In the case of treating a disease, such as cardiovascular disease, the desired response is inhibiting or reversing the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a nucleic acid molecule according to the invention for producing the desired response in a unit of weight or volume suitable for administration to a patient. The response can, for example, be measured by determining regression of cardiovascular disease and decrease of disease symptoms etc.

The doses of the nucleic acid molecule according to the invention administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. It will be apparent that the method of detection of the nucleic acid according to the invention facilitates the determination of an appropriate dosage for a subject in need of treatment.

In general, doses of the nucleic acid molecules herein disclosed of between 1 nM-1 μM generally will be formulated and administered according to standard procedures. Preferably doses can range from 1 nM-500 nM, 5 nM-200 nM, 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a nonhuman primate, cow, horse, pig, sheep, goat, dog, cat or rodent.

When administered, the pharmaceutical preparations of the invention are applied in pharmaceutically acceptable amounts and in pharmaceutically acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents e.g. statins. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Compositions may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “pharmaceutically acceptable carrier” in this context denotes an organic or inorganic ingredient, natural or synthetic, with which the active ingredient is combined to facilitate, for example, solubility and/or stability. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including acetic acid in a salt; citric acid in a salt; boric acid in a salt; and phosphoric acid in a salt. The pharmaceutical compositions also may contain, optionally, suitable preservatives.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Compositions suitable for oral administration may be presented as discrete units, such as capsules, tablets, lozenges, each containing a predetermined amount of the active compound.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a further preferred embodiment of the invention said pharmaceutical composition comprises at least one further, different, therapeutic agent.

In a preferred embodiment of the invention said further therapeutic agent is a statin.

Statins are commonly used to control cholesterol levels in subjects that have elevated LDL-C. Statins are effective in preventing and treating those subjects that are susceptible and those that have cardiovascular disease. The typical dosage of a statin is in the region 5 to 80 mg but this is dependent on the statin and the desired level of reduction of LDL-C required for the subject suffering from high LDL-C. However, expression and synthesis of HMG-CoA reductase, the target for statins, adapts in response to statin administration thus the beneficial effects of statin therapy are only temporary or limited after statin resistance is established.

Preferably said statin is selected from the group consisting of atorvastatin, fluvastatin, lovastatin, pitvastatin, pravastatin, rosuvastatin and simvastatin.

In a preferred embodiment of the invention said further therapeutic agent is ezetimibe.

Optionally, ezetimibe is combined with at least one statin, for example simvastatin.

In an alternative preferred embodiment of the invention said further therapeutic agent is selected from the group consisting of fibrates, nicotinic acid, cholestyramine.

In a further alternative preferred embodiment of the invention said further therapeutic agent is a therapeutic antibody, for example, evolocumab, bococizumab or alirocumab.

According to a further aspect of the invention there is provided a nucleic acid molecule or a pharmaceutical composition according to the invention for use in the treatment or prevention of a subject that has or is predisposed to hypercholesterolemia.

In a preferred embodiment of the invention said use is the treatment or prevention of diseases associated with hypercholesterolemia.

In a preferred embodiment of the invention said disease associated with hypercholesterolemia is selected from the group consisting of: stroke prevention, hyperlipidaemia, cardiovascular disease, atherosclerosis, coronary heart disease, aortic stenosis, cerebrovascular disease, peripheral arterial disease, hypertension, metabolic syndrome, type II diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

In a preferred embodiment of the invention said subject is a paediatric subject.

A paediatric subject includes neonates (0-28 days old), infants (1-24 months old), young children (2-6 years old) and prepubescent [7-14 years old] children.

In an alternative preferred embodiment of the invention said subject is an adult subject.

In a preferred embodiment of the invention the hypercholesterolemia is familial hypercholesterolemia.

In a preferred embodiment of the invention familial hypercholesterolemia is associated with elevated levels of apolipoprotein B expression.

In a preferred embodiment of the invention said subject is resistant to statin therapy.

According to a further aspect of the invention there is provided a method to treat a subject that has or is predisposed to hypercholesterolemia comprising administering an effective dose of a nucleic acid or a pharmaceutical composition according to the invention thereby treating or preventing hypercholesterolemia.

In a preferred method of the invention said subject is a paediatric subject.

In an alternative preferred method of the invention said subject is an adult subject.

In a preferred method of the invention the hypercholesterolemia is familial hypercholesterolemia.

In a preferred method of the invention familial hypercholesterolemia is associated with elevated levels of ApoB expression.

In a preferred method of the invention said subject is resistant to statin therapy.

According to a further aspect of the invention there is provided a treatment regimen for the diagnosis and treatment of hypercholesterolemia associated with elevated ApoB comprising:
i) obtaining a biological sample from a subject suspected on having or suspected of having hypercholesterolemia;
ii) contacting the sample with an antibody, or antibody fragment, specific for an apolipoprotein polypeptide;
iii) determining the concentration said apolipoprotein B polypeptide and LDL-C in said biological sample; and
iv) administering a nucleic acid molecule or pharmaceutical composition according to the invention if the LDL-C concentration is greater than 350 mg/dL.

Typically, in familial hypercholesterolemia disease the levels of LDL-C are 350-550 mg/dL in subjects that are heterozygous for a selected mutation in apolipoprotein B and 650-1000 mg/dL in those subjects carrying a homozygous mutation in apolipoprotein B. The normal levels of LDL-C are in the region 130 mg/dL.

Throughout the description and claims of this specification, the words “comprise” and “contain” and variations of the words, for example “comprising” and “comprises”, means “including but not limited to” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by Example only and with reference to the following figures:

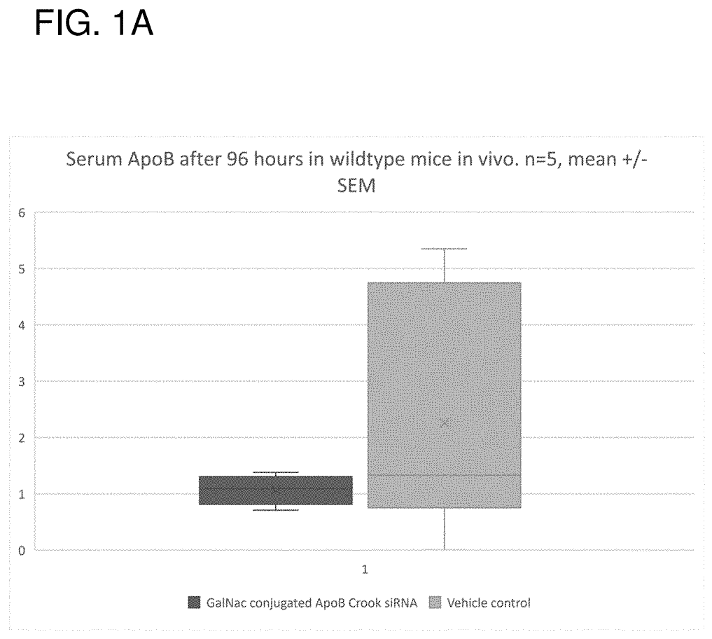

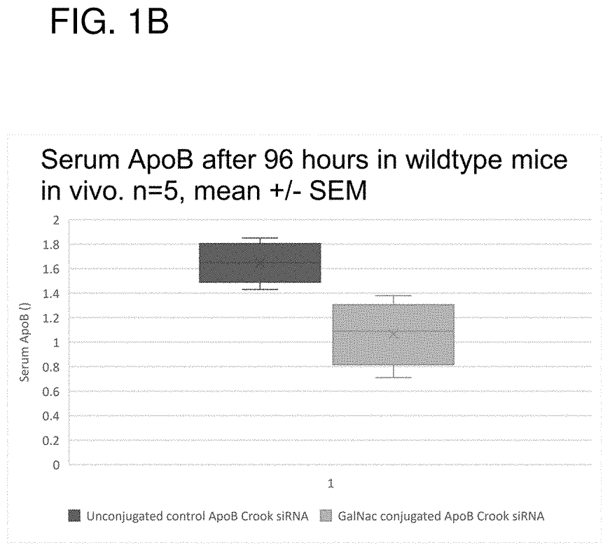

FIGS. 1A and 1B. Graphs illustrating in vivo Activity of GaINAc-conjugated Crook anti-mouse ApoB siRNA compared to control siRNA constructs.

(FIG. 1A) Plasma ApoB levels (micrograms/ml) from five adult male wild-type C57BL/6 mice, were measured 96 hours following administration of GaINAc-conjugated ApoB Crook siRNA (one treatment group) and compared with the control treatment group administered with saline. Statistical analysis was applied using the two-tailed paired T test algorithm. Results show a substantive reduction in mean plasma ApoB levels in mice treated with GaINAc-conjugated Crook siRNA, compared to control. However, it just fails significance (p=0.11), most likely due to small sample size and variation in ApoB levels between control animals;

(FIG. 1B) Plasma ApoB levels (micrograms/ml) from five adult male wild-type C57BL/6 mice, were measured 96 hours following administration of GaINAc-conjugated ApoB Crook siRNA (one treatment group) and compared with the control treatment group, administered with siRNA construct unconjugated (without GaINAc) ApoB Crook siRNA. Statistical analysis was applied using the two-tailed paired T test algorithm. Results show a highly significant reduction in plasma ApoB levels in this GaINAc-conjugated Crook siRNA treatment group when compared to control unconjugated siRNA with Crook (P=0.00435832).

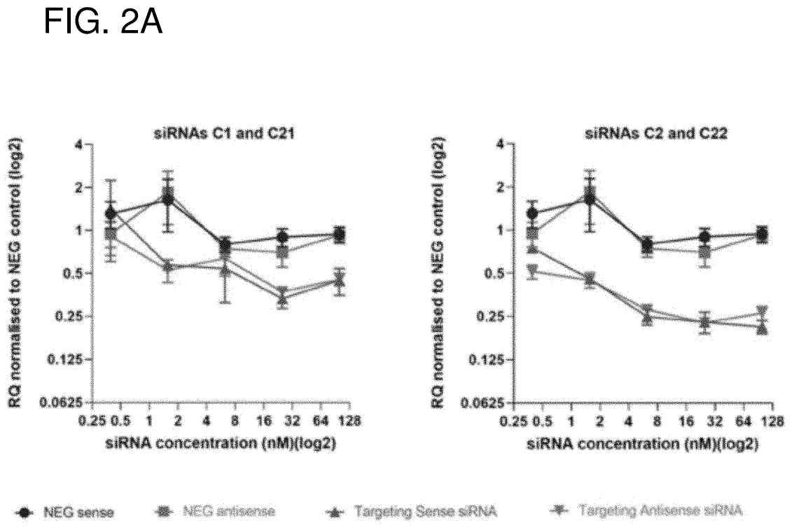

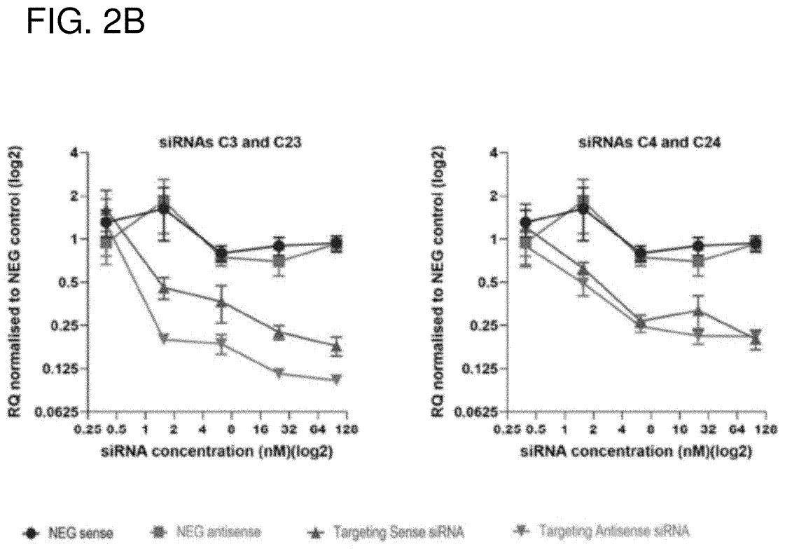

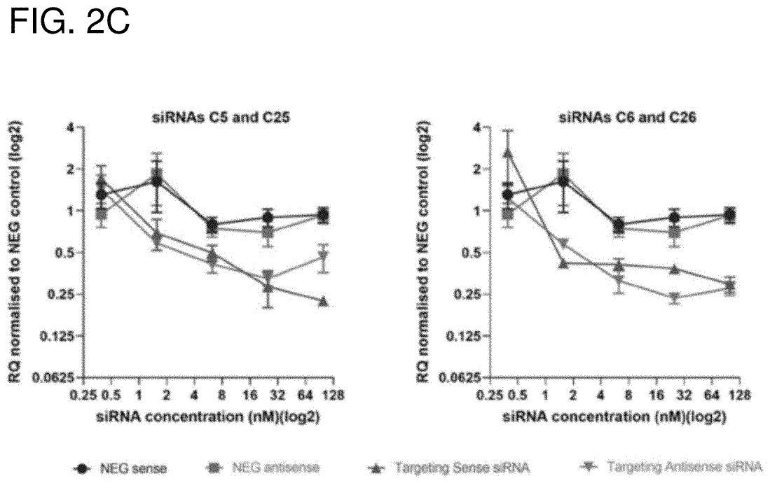

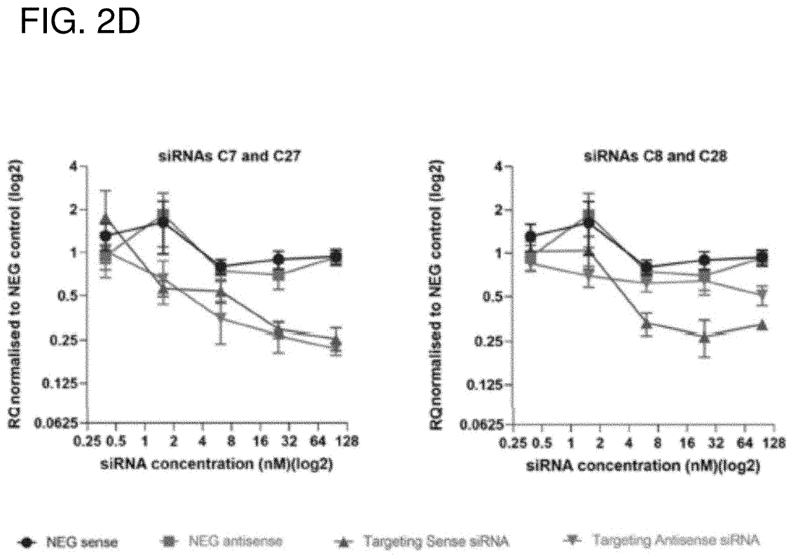

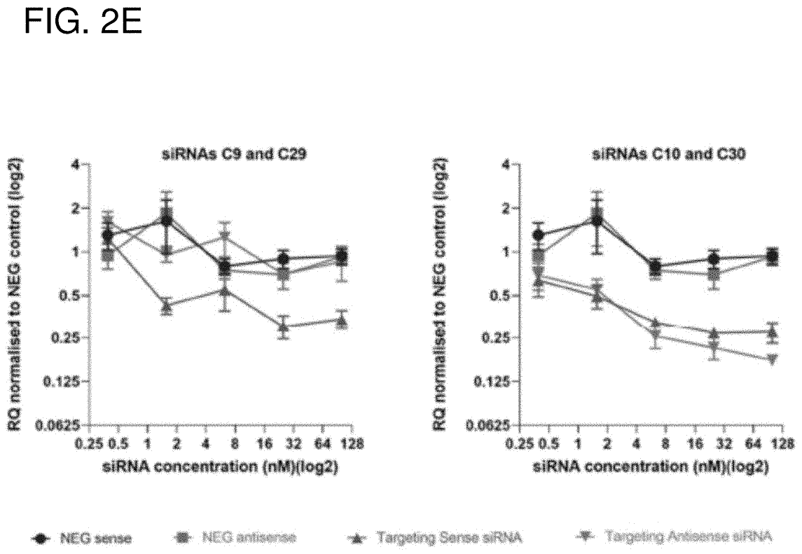

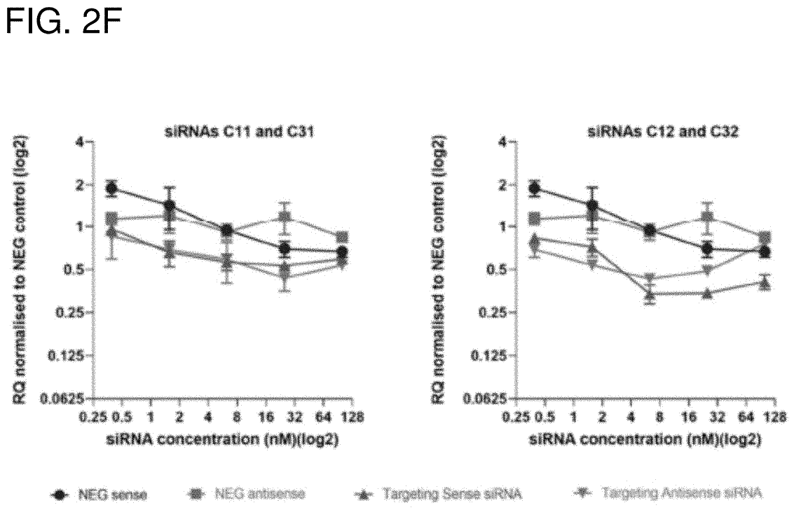

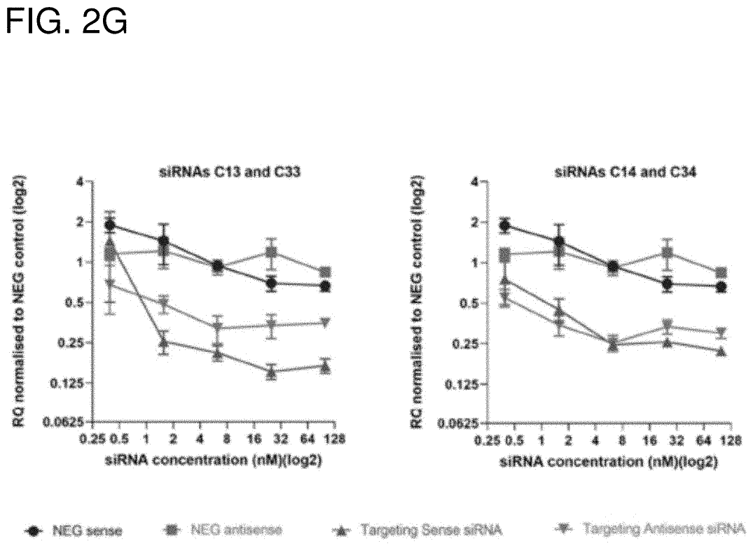

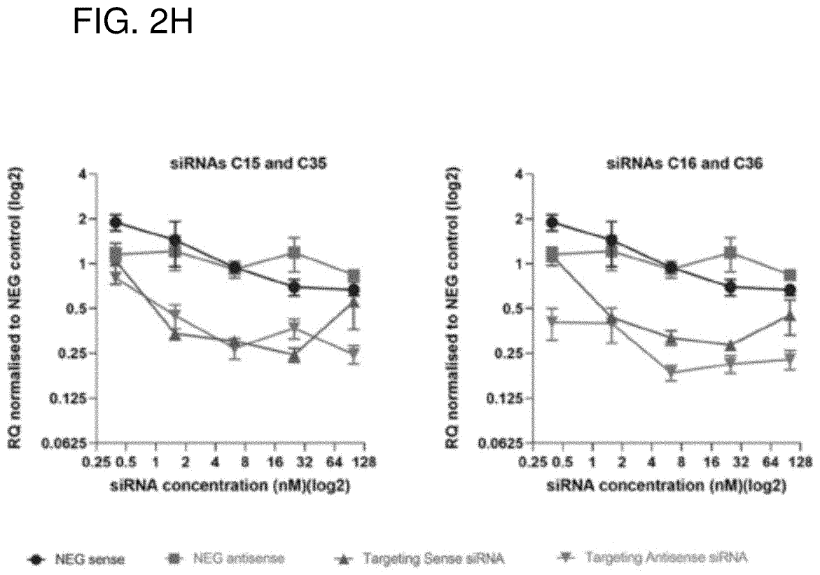

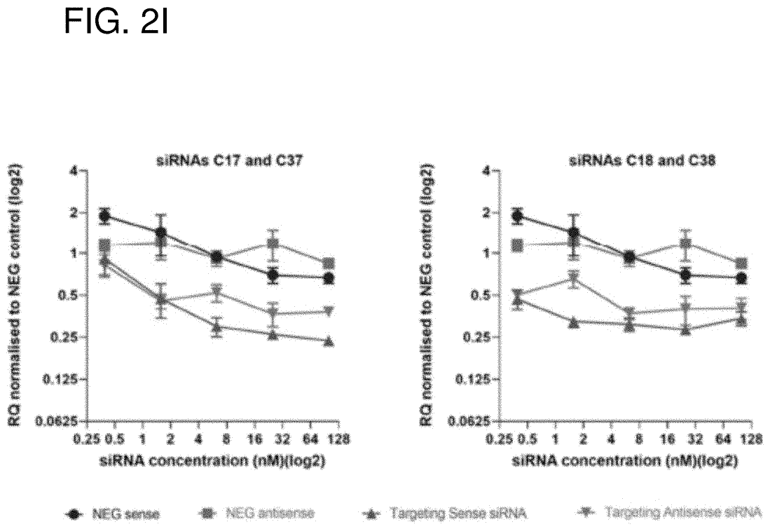

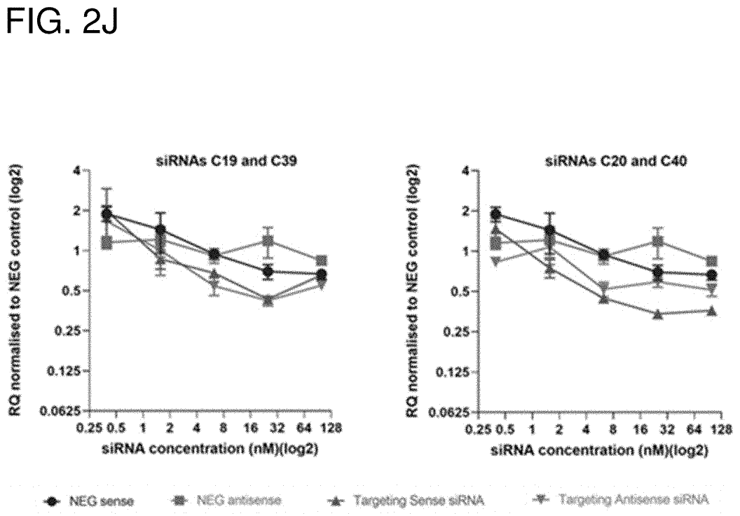

FIGS. 2A-2J. In vitro screen of 40 custom duplex Crook siRNAs (C1-C40) listed in Table 2. Graphs illustrate relative knock down of ApoB mRNA expression in HepG2 cells by each of the 40 crook siRNAs. Individual graphs present data from each siRNA sense and antisense pair; C1-C20 (sense strand); C21-40 (antisense strand) as shown in Table 2. Each of 40 crook siRNA molecules were reverse transfected into HepG2 cells (in quadruplicate) at five doses (100 nM, 25 nM, 6.25 nM, 1.56 nM and 0.39 nM) using the conditions identified in the assay development phase. 72 hours post transfection, cells were lysed and ApoB mRNA levels determined by duplex RT-qPCR. In order to calculate the relative knockdown of ApoB for each siRNA at each concentration, expression was first normalised to housekeeping reference gene GAPDH mRNA expression and then to the average ApoB expression across the five doses of the corresponding NEG control (Sense or Antisense). However, it should be noted that a decrease in ApoB expression was observed for the NEG sense control and so the knockdown of ApoB for siRNAs C1-C20 may be underestimated. ApoB knockdown efficiency of all 40 siRNAs are described in Table 3. C13 and C23 siRNAs show a knock-down efficiency greater than 85% (at 25 nM).

Table 3 was compiled from the in vitro ApoB mRNA expression data (FIG. 2 (a-d)) and shows ranking of Crook siRNAs (C1-C40) with highest knockdown performers at the top of the table. C13 and C23 siRNAs show a knock-down efficiency greater than 85% (at 25 nM).

### Materials and Methods

In Vivo Activity of GaINAc-Conjugated Crook Anti-Mouse ApoB siRNA

A triantennary GaINAc conjugate was attached to the passenger strand of the Crook-siRNA via phosphoramidate linkage in order to improve selective siRNA delivery to the liver.

Unconjugated (without GaINAc) and conjugated (GaINAc) versions of ApoB Crook-siRNA described below, were administered to adult male wild-type (WT) C57BL/6 mice by intravenous (IV) and sub-cutaneous (SC) routes, respectively, to investigate the relative plasma and tissue exposure. In addition, control GaINAc-conjugated unmodified siRNA (without Crook) consruct was compared.

In Vivo ApoB siRNA Constructs

For in vivo silencing of Apo B is a mouse the sequence below was used (corresponds to C10 in table 2 below. This was chosen because a similar sequence had been successful previously in vivo (Soutshek et al Nature 2004; 432:173-178).

Crook-siRNA 21mer-dsRNA Construct (1): Anti-Mouse ApoB-GaINAc

Sense Strand SEQ ID NO: 47 (Passenger SEQ ID NO: 1):

5′-GUCAUCACACUGAAUACCAAU-d(tcacctcatcccgcgaagc)-
3′-[Tri-GalNAC]

Antisense Strand SEQ ID NO: 100:

5′-AUUGGUAUUCAGUGUGAUGAC-3′

### Structure of Final GaINAc Conjugate:

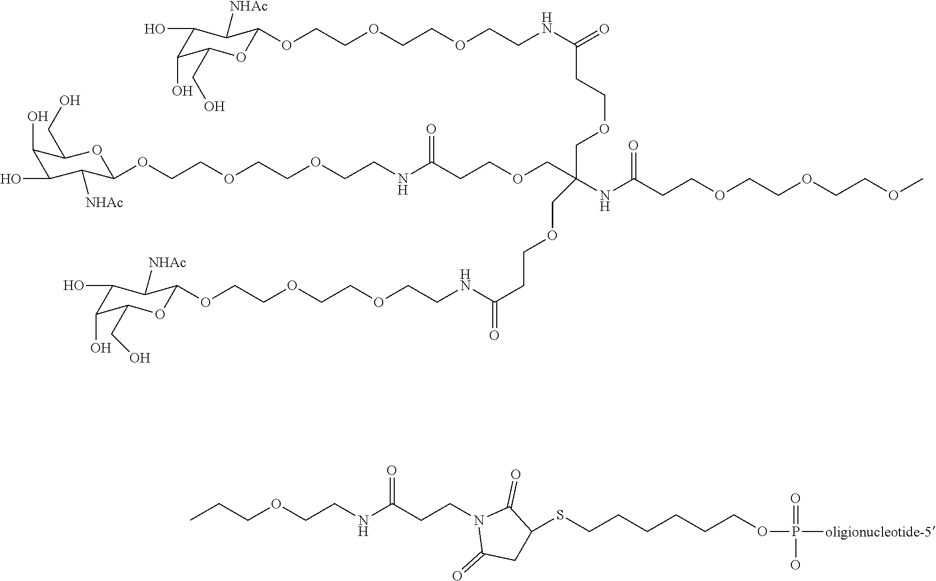

Crook-siRNA 21mer dsRNA Construct (2): Unconjugated Anti-Mouse ApoB

Sense Strand SEQ ID NO: 47 (Passenger SEQ ID NO: 1)

5′-GUCAUCACACUGAAUACCAAU-d(tcacctcatcccgcgaagc)-3

Antisense Strand (Guide)

(SEQ ID NO: 100)
5′-AUUGGUAUUCAGUGUGAUGAC-3

The rationale for dose selection was based on the following information published in the scientific literature:

The GaINAc conjugated siRNA is dosed subcutaneously at 5 mg/kg which is expected to produce the required level of gene silencing where the ED₈₀ of structurally related siRNAs have been reported as 2.5 mg/kg (Soutschek et al., 2004). These structurally related siRNAs were tolerated up to 25 mg/kg, single administration, in the mouse (Soutschek et al., 2004).

The unconjugated version of the sponsor's siRNA is administered at 50 mg/kg IV. This 10-fold increase in the IV compared to the SC dose is due to the unconjugated siRNA being less effective at targeting the liver. Additionally, it is reported by Soutschek et al (2004) that lower levels of RNA are measured in the liver following IV compared to SC administration. It is stated that slower release of the siRNA from the SC depot leads to prolonged exposure increasing the potential for receptor-ligand interactions and greater uptake into the tissue. Similar related siRNA has been well tolerated by mice at up to 50 mg/kg IV administered on 3 consecutive days (Nair et al. 2014). As a precaution a 15 minutes observation period is left between dosing the 1^{st} animal IV to determine if the test substance causes any adverse effects before the remaining animals are dosed.

The mouse is the species of choice because it is used as one of the toxicology species in the safety testing of the test substance. The mouse also possesses a very similar metabolic physiology to humans in relation to the therapeutic target of the Crook-siRNA preparations (ApoB). There is a considerable amount of published data available which are acceptable to the regulatory authorities for assessing the significance to man of data generated in this species.

### Animals

Sufficient C57BL/6 mice were obtained from an approved source to provide 20 healthy male animals (5 mice per treatment group). Animals are in the target weight range of 20 to 30 g at dosing. Mice are uniquely numbered by tail marking. Numbers are allocated randomly. Cages are coded by cards giving information including study number and animal number. The study room is identified by a card giving information including room number and study number. On receipt, all animals were examined for external signs of ill health. Unhealthy animals where be excluded from the study. The animals were acclimatized for a minimum period of 5 days. Where practicable, without jeopardizing the scientific integrity of the study, animals were handled as much as possible. A welfare inspection was performed before the start of dosing to ensure their suitability for the study.

The mice were kept in rooms thermostatically maintained at a temperature of 20 to 24° C., with a relative humidity of between 45 and 65%, and exposed to fluorescent light (nominal 12 hours) each day. Temperature and relative humidity are recorded on a daily basis. The facility is designed to give a minimum of 15 air-changes/hour. Except when in metabolism cages or recovering from surgery, mice were housed up to 5 per cage according to sex, in suitable solid floor cages, containing suitable bedding.

Cages conform to the ‘Code of Practice for the Housing and Care of Animals Bred, Supplied or Used for Scientific Purposes’ (Home Office, London, 2014). In order to enrich both the environment and the welfare of the animals, they were provided with wooden Aspen chew blocks and polycarbonate tunnels. The supplier provided certificates of analysis for each batch of blocks used. All animals will be allowed free access to 5LF2 EU Rodent Diet 14%. The diet supplier provided an analysis of the concentration of certain contaminants and some nutrients for each batch used. All animals were allowed free access to mains water from bottles attached to the cages. Periodic analysis of the mains supply is undertaken.

All procedures to be carried out on live animals as part of this study will be subject to provisions of United Kingdom National Law, the Animals (Scientific Procedures) Act 1986.

All animals were examined at the beginning and the end of the working day, to ensure that they are in good health. Any animal, which shows marked signs of ill health, were isolated. Moribund animals or those in danger of exceeding the severity limits imposed by the relevant Home Office License were killed.

### Materials and Methods for In Vivo Experiments

### Preparation of Formulations

Test substances were diluted in 0.9% saline to provided concentrations of 25 mg/mL and 0.6 mg/mL for the IV and SC doses of ApoB Crook-siRNA GaINAc-unconjugated and conjugate, respectively. The formulations were gently vortexed as appropriate until the test substances are fully dissolved. The resulting formulation(s) were assessed by visual inspection only and categorised accordingly:

(1) Clear solution

(2) Cloudy suspension, no particles visible

(3) Visible particles

After use, formulations were stored refrigerated nominally at 2-8° C.

Dosing Details Each animal received either a single IV dose of the ApoB Crook-siRNA—unconjugated or a single SC dose of the ApoB Crook-siRNA GaINAc— conjugate. The IV dose was administered as a bolus into the lateral tail vein at a volume of 2 mL/kg. The SC dose was administered into the subcutaneous space at a volume of 5 mL/kg.

| | | |
|---|---|---|
| Group1: | GalNAc-conjugated ApoB Crook siRNA | 5 mg/kg |
| | | dose |
| Group 2: | Unconjugated (without GalNAc) | 50 mg/kg |
| | ApoB Crook siRNA | dose |
| Group 3: | Saline control group | |

Body Weights

As a minimum, body weights were recorded the day after arrival and before dose administration. Additional determinations were made, if required.

### Sample Storage

Samples were uniquely labelled with information including, where appropriate: study number; sample type; dose group; animal number/Debra code; (nominal) sampling time; storage conditions. Samples were stored at <−50° C.

### Pharmacokinetic Investigation

### Designation of Dose Groups

Animals were assigned to dose groups as follows:

| | | | Dose | Number of |
|---|---|---|---|---|
| Dose | | | level | animals |
| Group | Dose route | Test Substance | mg/kg | Male |
| A | subcutaneous | ApoB Crook-siRNA | 5 | 5 |
| | | GalNAc-conjugate | | |
| B | subcutaneous | Saline control | 0 | 5 |
| C | Intravenous | ApoB Crook-siRNA | 50 | 5 |
| | (bolus) | unconjugated | | |
| D | Intravenous | Saline control | 0 | 5 |
| | (bolus) | | | |

### Blood Sampling

Serial blood samples of (nominally 100 μL, dependent on bodyweight) were collected by tail nick at the following times: 0, 48- and 96-hours post dose. Animals were terminally anaesthetised using sodium pentobarbitone and a final sample (nominally 0.5 mL) was collected by cardiac puncture.

Blood samples were collected in to a K2EDTA microcapillary tube (tail nick) or a K2EDTA blood tube (cardiac puncture) and placed on ice until processed. Blood was centrifuged (1500 g, 10 min, 4° C.) to produce plasma for analysis. The bulk plasma was divided into two aliquots of equal volume. The residual blood cells were discarded.

| Scheduled Collection | Acceptable Time | |
|---|---|---|
| Time | Range | |
| 0-15 minutes | ±1 | minute |
| 16-30 minutes | ±2 | minutes |
| 31-45 minutes | ±3 | minutes |
| 46-60 minutes | ±4 | minutes |
| 61 minutes- | ±5 | minutes |
| 2 hours | | |
| 2 hours 1 minute- | ±10 | minutes |
| 8 hours | | |
| 8 hours 1 minute- | ±15 | minutes |
| 12 hours | | |
| 12 hours | ±30 | minutes |
| onwards | | |

Where a scheduled collection time is outside the acceptable range, the actual blood collection time was reported for inclusion in any subsequent PK analysis

### Animal Fate

Animals were anaesthetised via an intraperitoneal injection of Sodium Pentobarbitone prior to terminal blood sampling and sacrificed by perfusion and exsanguination.

A full body perfusion was performed, all animals were flushed with Heparinised Saline Solution at a rate 4 ml/min for 5 minutes (approximately 20 mL total flush). Death was confirmed by the absence of breathing, heartbeat and blood flow. Animal carcasses were retained for tissue collection.

### Tissue Collection

The liver was removed from all animals (Groups A-D) and placed into a pre-weighed tube. The tissue samples were homogenised with 5 parts RNAlater to 1 part tissue using the UltraTurrax homogenisation probe. The following tissues were excised from animals in ApoB treated groups (Groups A & C) and placed into a pre-weighed pot:
Spleen
Brain
Heart
Lung Lobes
Skin (Inguinal region ca. 25 mm²)

Following collection, the external surface of the tissues is rinsed with PBS and gently patted dry using a tissue. Tissues are initially placed on wet ice until weighed and then tissues were snap frozen on dry ice prior to storage. Tissues are stored at <−50° C. (nominally −80° C.).

### Immunoassay for APOB

Plasma ApoB levels were measured via enzyme-linked immunosorbent assay (ELISA) using the commercial mouse ApoB detection kit from Elabscience Biotechnology Inc. (catalogue number E-EL-M0132). Plasma samples were stored at −80° C. prior to analysis, thawed on ice and centrifuged at 13,000 rpm for 5 minutes prior to aliquots being diluted in Assay Buffer and applied to the ELISA plate. The ApoB assay kit uses a sandwich ELISA yielding a colorimetric readout, measured at OD450. Samples from each animal at specific time points (0 hours and 96 hours) were assayed in duplicate and measurements were recorded as micrograms ApoB per ml of plasma based on the standard curve reagents supplied with the kit. All data points were measured with a coefficient of variation <20%.

In vitro Screening of ApoB Crook siRNA

### HepG2 Reverse Transfection

A description of the custom library evaluated in this study is provided in Table 2. Custom duplex siRNAs synthesized by Horizon Discovery were resuspended in UltraPure DNase and RNase free water to generate a stock solution of 10 μM.
Stock siRNAs were dispensed into 4×384-well assay plates. On each assay plate, 10 Custom siRNAs and 3 controls (POS ApoB, NEG sense and NEG antisense) were dispensed to generate five-point four-fold dilution series from a top final concentration in the assay plate of 100 nM. ON-TARGETplus Non-Targeting and ApoB siRNAs controls were dispensed to give a final concentration of 25 nM.
Lipofectamine RNAiMAX (ThermoFisher #13778075) was diluted in OptiMEM media before 10 μL of the Lipfectamine RNAiMAX:OptiMEM solution was added per well to the assay plate. The final volume of RNAiMAX per well was 0.08 μL.
The lipid-siRNA mix was incubated 30 min at room temperature before being added to the cells.
HepG2 cells were diluted in assay media (MEM GlutaMAX (GIBCO) 10% FBS 1% Pen/Strep) before 4,000 HepG2 cells were seeded into each well of the assay plate in 40 μL volume. Quadruplicate technical replicates were seeded per assay condition.
The plates were incubated 72 h at 37° C., 5% CO₂ in a humidified atmosphere, prior to assessment of the cells.

### ApoB/GAPDH Duplex RT-qPCR

72 h post-transfection, cells were processed for RT-qPCR read-out using the Cells-to-CT 1-step TaqMan Kit (Invitrogen, 4391851C). Briefly, cells were washed with 50 μl cold PBS and then lysed in 20 μl Lysis solution containing DNase I. After 5 min, lysis was stopped by addition of 2 μl STOP Solution for 2 min.
For the RT-qPCR analysis, 3 μl of lysate was dispensed per well into 384-well PCR plate as template in an 11 μl RT-qPCR reaction volume.
RT-qPCR was performed using the ThermoFisher TaqMan Fast Virus1-Step Master Mix (#4444434) with TaqMan probes for GAPDH (VIC #4448486) and ApoB (FAM #4351368).
RT-qPCR was performed using a QuantStudio 6 thermocycling instrument (Applied BioSystems).
Relative quantification (RQ) was determined using the ΔΔCT method, where GAPDH was used as internal control and expression changes normalized to the reference sample (either NEG sense or NEG antisense siRNA treated cells).

### Statistics

For all assays in this project, four technical replicates were obtained for each data point.
Mean and Standard Error of the Mean (SEM) were calculated using Excel or Graphpad Prism.
All graphs were generated using Graphpad Prism.

TABLE 1
ApoB crook siRNA sequences and corresponding SEQ ID NOs.

| SEQ | | SEQ | | |
|---|---|---|---|---|
| ID | Sense | ID | Antisense | Start |
| NO | strand base sequence | NO | strand base sequence | NM_009693.2 |
| 5 | GAGGUGUAUGGCUUCAACCCU | 58 | AGGGUUGAAGCCAUACACCUC | 403 |
| 6 | AGGUGUAUGGCUUCAACCCUG | 59 | CAGGGUUGAAGCCAUACACCU | 404 |
| 7 | GGUGUAUGGCUUCAACCCUGA | 60 | UCAGGGUUGAAGCCAUACACC | 405 |
| 8 | GUGUAUGGCUUCAACCCUGAG | 61 | CUCAGGGUUGAAGCCAUACAC | 406 |
| 9 | GUAUGGCUUCAACCCUGAGGG | 62 | CCCUCAGGGUUGAAGCCAUAC | 408 |
| 10 | AUGGCUUCAACCCUGAGGGCA | 63 | UGCCCUCAGGGUUGAAGCCAU | 410 |
| 11 | UGGCUUCAACCCUGAGGGCAA | 64 | UUGCCCUCAGGGUUGAAGCCA | 411 |
| 12 | CUGAACAUCAAGAGGGGCAUC | 65 | GAUGCCCCUCUUGAUGUUCAG | 562 |
| 13 | UGAACAUCAAGAGGGGCAUCA | 66 | UGAUGCCCCUCUUGAUGUUCA | 563 |
| 14 | GAUACCGUGUAUGGAAACUGC | 67 | GCAGUUUCCAUACACGGUAUC | 640 |
| 15 | UACCGUGUAUGGAAACUGCUC | 68 | GAGCAGUUUCCAUACACGGUA | 642 |
| 16 | GUCCAGCCCCAUCACUUUACA | 69 | UGUAAAGUGAUGGGGCUGGAC | 1221 |
| 17 | CAGCCCCAUCACUUUACAAGC | 70 | GCUUGUAAAGUGAUGGGGCUG | 1224 |
| 18 | AGCCCCAUCACUUUACAAGCC | 71 | GGCUUGUAAAGUGAUGGGGCU | 1225 |
| 19 | GCCCCAUCACUUUACAAGCCU | 72 | AGGCUUGUAAAGUGAUGGGGC | 1226 |
| 20 | CUUUACAAGCCUUGGUUCAGU | 73 | ACUGAACCAAGGCUUGUAAAG | 1235 |
| 21 | ACAAGCCUUGGUUCAGUGUGG | 74 | CCACACUGAACCAAGGCUUGU | 1239 |
| 22 | AAGCCUUGGUUCAGUGUGGAC | 75 | GUCCACACUGAACCAAGGCUU | 1241 |
| 23 | UCACAUCCUCCAGUGGCUGAA | 76 | UUCAGCCACUGGAGGAUGUGA | 1278 |
| 24 | AAAUAGAAGGGAAUCUUAUAU | 77 | AUAUAAGAUUCCCUUCUAUUU | 2063 |
| 25 | UAGAAGGGAAUCUUAUAUUUG | 78 | CAAAUAUAAGAUUCCCUUCUA | 2066 |
| 26 | GAAGGGAAUCUUAUAUUUGAU | 79 | AUCAAAUAUAAGAUUCCCUUC | 2068 |
| 27 | GGAAUCUUAUAUUUGAUCCAA | 80 | UUGGAUCAAAUAUAAGAUUCC | 2072 |
| 28 | GAGUUUGUGACAAAUAUGGGC | 81 | GCCCAUAUUUGUCACAAACUC | 2746 |
| 29 | GUUUGUGACAAAUAUGGGCAU | 82 | AUGCCCAUAUUUGUCACAAAC | 2748 |
| 30 | GUGACAAAUAUGGGCAUCAUC | 83 | GAUGAUGCCCAUAUUUGUCAC | 2752 |
| 31 | AGAUGAACACCAACUUCUUCC | 84 | GGAAGAAGUUGGUGUUCAUCU | 2801 |
| 32 | GAUGAACACCAACUUCUUCCA | 85 | UGGAAGAAGUUGGUGUUCAUC | 2802 |
| 33 | UGAACACCAACUUCUUCCACG | 86 | CGUGGAAGAAGUUGGUGUUCA | 2804 |
| 34 | GAACACCAACUUCUUCCACGA | 87 | UCGUGGAAGAAGUUGGUGUUC | 2805 |
| 35 | ACACCAACUUCUUCCACGAGU | 88 | ACUCGUGGAAGAAGUUGGUGU | 2807 |
| 36 | CACCAACUUCUUCCACGAGUC | 89 | GACUCGUGGAAGAAGUUGGUG | 2808 |
| 37 | CAAAUGGACUCAUCUGCUACA | 90 | UGUAGCAGAUGAGUCCAUUUG | 3547 |
| 38 | GGACUCAUCUGCUACAGCUUA | 91 | UAAGCUGUAGCAGAUGAGUCC | 3552 |
| 39 | UCUGUGGGAUUCCAUCUGCCA | 92 | UGGCAGAUGGAAUCCCACAGA | 4075 |
| 40 | AUUCCAUCUGCCAUCUCGAGA | 93 | UCUCGAGAUGGCAGAUGGAAU | 4083 |
| 41 | ACAAUUUGAUCAGUAUAUUAA | 94 | UUAAUAUACUGAUCAAAUUGU | 6636 |
| 42 | CAAUUUGAUCAGUAUAUUAAA | 95 | UUUAAUAUACUGAUCAAAUUG | 6637 |
| 43 | UAAAUCAAGUGUCAUCACACU | 96 | AGUGUGAUGACACUUGAUUUA | 10116 |
| 44 | AUCAAGUGUCAUCACACUGAA | 97 | UUCAGUGUGAUGACACUUGAU | 10119 |
| 45 | UCAAGUGUCAUCACACUGAAU | 98 | AUUCAGUGUGAUGACACUUGA | 10120 |
| 46 | CAAGUGUCAUCACACUGAAUU | 99 | AAUUCAGUGUGAUGACACUUG | 10121 |
| 47 | GUCAUCACACUGAAUACCAAU | 100 | AUUGGUAUUCAGUGUGAUGAC | 10126 |
| 48 | UCAUCACACUGAAUACCAAUG | 101 | CAUUGGUAUUCAGUGUGAUGA | 10127 |
| 49 | CAUCACACUGAAUACCAAUGC | 102 | GCAUUGGUAUUCAGUGUGAUG | 10128 |
| 50 | UAACACUAAGAACCAGAAGAU | 103 | AUCUUCUGGUUCUUAGUGUUA | 10959 |
| 51 | AUUGGGAAGAAGAGGCAGCUU | 104 | AAGCUGCCUCUUCUUCCCAAU | 12167 |
| 52 | GAUUGAUUGACCUGUCCAUUC | 105 | GAAUGGACAGGUCAAUCAAUC | 13478 |
| 53 | UGAUUGACCUGUCCAUUCAAA | 106 | UUUGAAUGGACAGGUCAAUCA | 13481 |
| 54 | GAUUGACCUGUCCAUUCAAAA | 107 | UUUUGAAUGGACAGGUCAAUC | 13482 |
| 55 | GACCUGUCCAUUCAAAACUAC | 108 | GUAGUUUUGAAUGGACAGGUC | 13486 |
| 56 | ACCUGUCCAUUCAAAACUACC | 109 | GGUAGUUUUGAAUGGACAGGU | 13487 |
| 57 | CUGUCCAUUCAAAACUACCAC | 110 | GUGGUAGUUUUGAAUGGACAG | 13489 |
| 111 | CAGCACCUAGCUGGAAAGUUA | 112 | UAACUUUCCAGCUAGGUGCUG | |
| 113 | CUCCAUGGAAUUUAAGUAUGA | 114 | UCAUACUUAAAUUCCAUGGAG | |
| 115 | UUCCCUGAAGUUGAUGUGUUA | 116 | UAACACAUCAACUUCAGGGAA | |
| 117 | GUCCAAUAAGAUCAAUAGCAA | 118 | UUGCUAUUGAUCUUAUUGGAC | |
| 119 | AACUCUCAAACCCUAAGAUUA | 120 | UAAUCUUAGGGUUUGAGAGUU | |
| 121 | UCGGAACAAUCCUCAGAGUUA | 122 | UAACUCUGAGGAUUGUUCCGA | |
| 123 | AAGCAAGAACUUAAUGGAAAU | 124 | AUUUCCAUUAAGUUCUUGCUU | |
| 125 | GGCCAUUAGGCAAAUUGAUGA | 126 | UCAUCAAUUUGCCUAAUGGCC | |

TABLE 2
A library of 40 duplex siRNAs was synthesized by Horizon Discovery. The table
shows the sequences of both strands of RNA for each siRNA. The following DNA
sequence (dTdCdAdCdCdTdCdAdTdCdCdCdGdCdGdAdAdGdC) was appended to the 3′ end
of either the sense strand (siRNAs C1 to C20, thereafter referred to as sense
siRNAs) or the antisense strand (siRNAs C21 to C40, thereafter referred to
as antisense siRNAs).

| siRNA ID | | | |
|---|---|---|---|
| Sense | Antisense | Sense sequence (5′-3′) | Antisense sequence (5′-3′) |
| C1 | C21 | UAGAAGGGAAUCUUAUAUUUG | CAAAUAUAAGAUUCCCUUCUA |
| | | (SEQ ID NO: 25) | (SEQ ID NO: 78) |
| C2 | C22 | CACCAACUUCUUCCACGAGUC | GACUCGUGGAAGAAGUUGGUG |
| | | (SEQ ID NO: 36) | (SEQ ID NO: 89) |
| C3 | C23 | GGUGUAUGGCUUCAACCCUGA | UCAGGGUUGAAGCCAUACACC |
| | | (SEQ ID NO: 7) | (SEQ ID NO: 60) |
| C4 | C24 | GACCUGUCCAUUCAAAACUAC | GUAGUUUUGAAUGGACAGGUC |
| | | (SEQ ID NO: 55) | (SEQ ID NO: 108) |
| C5 | C25 | UACCGUGUAUGGAAACUGCUC | GAGCAGUUUCCAUACACGGUA |
| | | (SEQ ID NO: 15) | (SEQ ID NO: 68) |
| C6 | C26 | GCCCCAUCACUUUACAAGCCU | AGGCUUGUAAAGUGAUGGGGC |
| | | (SEQ ID NO: 19) | (SEQ ID NO: 72) |
| C7 | C27 | GAUUGAUUGACCUGUCCAUUC | GAAUGGACAGGUCAAUCAAUC |
| | | (SEQ ID NO: 52) | (SEQ ID NO: 105) |
| C8 | C28 | GAGGUGUAUGGCUUCAACCCU | AGGGUUGAAGCCAUACACCUC |
| | | (SEQ ID NO: 5) | (SEQ ID NO: 58) |
| C9 | C29 | UCUGUGGGAUUCCAUCUGCCA | UGGCAGAUGGAAUCCCACAGA |
| | | (SEQ ID NO: 39) | (SEQ ID NO: 92) |
| C10 | C30 | GUCAUCACACUGAAUACCAAU | AUUGGUAUUCAGUGUGAUGAC |
| | | (SEQ ID NO: 47) | (SEQ ID NO: 100) |
| C11 | C31 | GUGACAAAUAUGGGCAUCAUC | GAUGAUGCCCAUAUUUGUCAC |
| | | (SEQ ID NO: 30) | (SEQ ID NO: 83) |
| C12 | C32 | ACCUGUCCAUUCAAAACUACC | GGUAGUUUUGAAUGGACAGGU |
| | | (SEQ ID NO: 56) | (SEQ ID NO: 109) |
| C13 | C33 | CAGCACCUAGCUGGAAAGUUA | UAACUUUCCAGCUAGGUGCUG |
| | | (SEQ ID NO: 111) | (SEQ ID NO: 112) |
| C14 | C34 | CUCCAUGGAAUUUAAGUAUGA | UCAUACUUAAAUUCCAUGGAG |
| | | (SEQ ID NO: 113) | (SEQ ID NO: 114) |
| C15 | C35 | UUCCCUGAAGUUGAUGUGUUA | UAACACAUCAACUUCAGGGAA |
| | | (SEQ ID NO: 115) | (SEQ ID NO: 116) |
| C16 | C36 | GUCCAAUAAGAUCAAUAGCAA | UUGCUAUUGAUCUUAUUGGAC |
| | | (SEQ ID NO: 117) | (SEQ ID NO: 118) |
| C17 | C37 | AACUCUCAAACCCUAAGAUUA | UAAUCUUAGGGUUUGAGAGUU |
| | | (SEQ ID NO: 119) | (SEQ ID NO: 120) |
| C18 | C38 | UCGGAACAAUCCUCAGAGUUA | UAACUCUGAGGAUUGUUCCGA |
| | | (SEQ ID NO: 121) | (SEQ ID NO: 122) |
| C19 | C39 | AAGCAAGAACUUAAUGGAAAU | AUUUCCAUUAAGUUCUUGCUU |
| | | (SEQ ID NO: 123) | (SEQ ID NO: 124) |
| C20 | C40 | GGCCAUUAGGCAAAUUGAUGA | UCAUCAAUUUGCCUAAUGGCC |
| | | (SEQ ID NO: 125) | (SEQ ID NO: 126) |

### EXAMPLE 1

A pilot in vivo mouse experiment was performed to assess activity of GaINAc-conjugated Crook anti-mouse ApoB siRNA compared to control siRNA constructs. Conjugated (GaINAc) and unconjugated (without GaINAc) versions of ApoB Crook siRNA (sequence C10 in Table 2; Covance) were administered to adult male wild-type (WT) C57BL/6 mice by sub-cutaneous (SC) and intravenous (IV) routes, respectively described previously in Material & Methods section.

Blood plasma ApoB was measured by ELISA (described earlier) at time 0 (prior to administration of siRNA construct) and at 96 hours following siRNA construct administration, as indicated in the four Treatment groups (5 mice per group) as detailed above under Dosing Details.

Plasma ApoB levels (micrograms/ml) from 5 mice in each treatment group, were used to calculate a mean ApoB value+/− standard error of the mean (SEM). Change in plasma ApoB level after 96 hours following SC administration of GaINAc-conjugated Crook siRNA was compared to levels in mice receiving either control (i) vehicle saline, or (ii) unconjugated siRNA with Crook. Statistical analysis was applied using the two-tailed paired T test algorithm.

With reference to FIG. 1 (a), plasma ApoB levels (micrograms/ml) of mice 96 hours following treatment with GaINAc-conjugated ApoB Crook siRNA were compared with the control treatment group administered with saline. Statistical analysis was applied using the two-tailed paired T test algorithm. Results show a substantive reduction in mean plasma ApoB levels in mice treated with GaINAc-conjugated Crook siRNA, compared to control. However, it just fails significance (p=0.11), most likely due to small sample size and variation in ApoB levels between control animals.

With reference to FIG. 1 (b), plasma ApoB levels (micrograms/ml) measured 96 hours following administration of GaINAc-conjugated ApoB Crook siRNA were compared to the control group, treated with siRNA construct unconjugated (without GaINAc) ApoB Crook siRNA. Statistical analysis was applied using the two-tailed paired T test algorithm.

Results show a highly significant reduction in plasma ApoB levels in this GaINAc-conjugated Crook siRNA treatment group when compared to control unconjugated siRNA with Crook (P=0.00435832).

Importantly, the selected ApoB Crook siRNA sequence (C10 in Table 2; Covance) used in this pilot in vivo experiment was performed prior to the in vitro ApoB Crook siRNA screen. Our subsequent in vitro data (Example 2) shows that there are other siRNA sequences with greater ApoB mRNA knockdown (KD) efficiency (Table 3) eg. C23 gives 89% KD at 25 nM when compared to C10 (74%).

### EXAMPLE 2

With reference to FIG. 2 (a-d), an arrayed RNAi screen in HepG2 cells was performed to evaluate a custom library of 40 “crook” siRNAs targeting the human ApoB gene (Table 2). All siRNAs in this library possess a DNA extension (or “crook”) appended to the sense RNA strand (sense siRNA) or to the antisense RNA strand (antisense siRNA).

Prior to performing the screen, suitable conditions for HepG2 reverse transfection were identified. First, siRNAs targeting essential genes were used to evaluate a number of transfection conditions before the selected condition was taken forward to knockdown expression of ApoB using an ON-TARGETplus siRNA and assess the molecular detection tools for analysis of ApoB gene and protein expression. Homogeneous Time-Resolved Fluorescence (HTRF) and Duplex Real-Time quantitative PCR (RT-qPCR) assays were developed to quantify ApoB expression change at the protein and mRNA levels, respectively. In the Screening phase, the 40 custom crook siRNAs were assessed over a five-point dose range. 72 h post transfection, ApoB expression was evaluated by Duplex RT-qPCR. With a few notable exceptions, the knockdown of ApoB expression was similar between the sense and antisense siRNAs sharing the same RNA sequence, when the data is normalised to its relevant negative control (NEG sense for sense siRNAs and NEG antisense for the antisense siRNAs). However, it should be noted that a decrease in ApoB expression was observed for the NEG sense control and so the knockdown of ApoB for siRNAs C1-C20 may be underestimated.

HepG2 cells were reverse transfected with a library of 40 custom crook siRNAs (20 sense siRNAs and 20 antisense siRNAs) alongside the siRNA controls using conditions identified in the assay development phase. 72 h post transfection, ApoB mRNA levels in transfected cells were quantified by duplex RT-qPCR, normalizing the ApoB mRNA levels to the levels of the housekeeping reference gene GAPDH mRNA (FIG. 2 (a-d)).

As the assessment of the 40 custom crook siRNA molecules covered a number of assay plates, in order to be able to perform an assay QC step, each plate contained a number of controls. These included the ON-TARGETplus (OT+) siRNAs targeting ApoB and a matched non-targeting control assessed at 25 nM as well as the Negative controls for the sense and antisense siRNAs (NEG sense and NEG antisense, respectively) and the Argonaute control ApoB siRNA (POS ApoB).

With reference to FIG. 2 (a-d), a dose dependent decrease in ApoB expression with increasing siRNA concentration was observed for the majority of the siRNA tested, however the level of knockdown observed differed between the siRNAs, as anticipated. Knockdown of ApoB by the targeting siRNAs was greater than for the NEG controls for all siRNAs tested, with the exception of siRNAs C28 and C29, where limited knockdown was observed.

With reference to Table 3, when sense and antisense siRNAs targeting the same RNA sequence are compared the knockdown was similar. Four siRNA pairs appeared to show a differential in knockdown efficiency between the sense and antisense siRNA. These were C3-C23, C8-C28, C9-C29, and C13-C33. For all of these, except C3-C23, the sense siRNA appeared to be more efficient than the antisense siRNA, however for C8-C28 and C9-C29, the antisense siRNA did not appear to knockdown ApoB expression.

Overall based on the ApoB mRNA level following treatment with 25 nM siRNA, the following siRNAs display the best knock-down efficiency: the sense crook siRNAs C3 and C13 and the antisense crook siRNAs C23, C24, C30 and C36. C13 and C23 are the only two siRNAs showing a knock-down efficiency greater than 85% at this dose; see Table 3.

TABLE 3
In vitro activity of ApoB Crook siRNAs (C1-C40) in HepG2 cells.
Each siRNA is ranked according to ApoB mRNA knockdown (KD)
performance, with highest KD at the top of the table.

| siRNA | | | Crook position: | |
|---|---|---|---|---|
| Construct | ApoB mRNA Knockdown | | Sense (S) | NM_000384 |
| Code | 6.25 nM | 25 nM | Anti-sense (A) | Start position |
| C23 | 0.18 (82%) | 0.11 (89%) | A | 423 |
| C13 | 0.20 (80%) | 0.15 (85%) | S | 6964 |
| C36 | 0.18 (82%) | 0.20 (80%) | A | 9006 |
| C24 | 0.24 (76%) | 0.20 (80%) | A | 13693 |
| C30 | 0.25 (75%) | 0.20 (80%) | A | 10168 |
| C2 | 0.25 (75%) | 0.22 (78%) | S | 2835 |
| C22 | 0.27 (73%) | 0.22 (78%) | A | 2835 |
| C3 | 0.36 (64%) | 0.22 (78%) | S | 423 |
| C26 | 0.31 (69%) | 0.23 (77%) | A | 1253 |
| C15 | 0.29 (71%) | 0.24 (76%) | S | 11500 |
| C14 | 0.24 (76%) | 0.25 (75%) | S | 10497 |
| C17 | 0.29 (71%) | 0.25 (75%) | S | 8567 |
| C35 | 0.26 (74%) | 0.36 (64%) | A | 11500 |
| C10 | 0.32 (68%) | 0.26 (74%) | S | 10168 |
| C8 | 0.32 (68%) | 0.26 (74%) | S | 430 |
| C27 | 0.34 (66%) | 0.26 (74%) | A | 13685 |
| C16 | 0.30 (70%) | 0.28 (72%) | S | 9006 |
| C18 | 0.30 (70%) | 0.28 (72%) | S | 3347 |
| C5 | 0.49 (51%) | 0.28 (72%) | S | 669 |
| C7 | 0.52 (48%) | 0.28 (72%) | S | 13685 |
| C9 | 0.54 (46%) | 0.30 (70%) | S | 4102 |
| C4 | 0.26 (74%) | 0.31 (69%) | S | 13693 |
| C25 | 0.40 (60%) | 0.32 (68%) | A | 669 |
| C20 | 0.44 (56%) | 0.33 (67%) | S | 4102 |
| C1 | 0.75 (25%) | 0.32 (68%) | S | 2093 |
| C34 | 0.25 (75%) | 0.33 (67%) | A | 10497 |
| C33 | 0.31 (69%) | 0.33 (67%) | A | 6964 |
| C12 | 0.33 (67%) | 0.33 (67%) | S | 13694 |
| C37 | 0.51 (49%) | 0.36 (64%) | A | 8567 |
| C6 | 0.40 (60%) | 0.37 (63%) | S | 1253 |
| C21 | 0.62 (38%) | 0.36 (64%) | A | 2093 |
| C38 | 0.36 (64%) | 0.39 (61%) | A | 3347 |
| C39 | 0.53 (47%) | 0.41 (59%) | A | 10450 |
| C31 | 0.57 (43%) | 0.43 (57%) | A | 2778 |
| C19 | 0.67 (33%) | 0.42 (58%) | S | 10450 |
| C32 | 0.43 (57%) | 0.48 (52%) | A | 13694 |
| C11 | 0.55 (45%) | 0.52 (48%) | S | 2778 |
| C40 | 0.51 (49%) | 0.58 (42%) | A | 4102 |
| C28 | 0.60 (40%) | 0.63 (37%) | A | 430 |
| C29 | 1.25 (−25%) | 0.69 (31%) | A | 4102 |

### REFERENCES

Nair, J. K., Willoughby, J. L., Chan, A., Charisse, K., Alam, M. R., Wang, a, Hoekstra, M., Kandasamy, P., Kel'in, A. V., Milstein, S. and Taneja, N., 2014. Multivalent N-acetylgalactosamine-conjugated siRNA localizes in hepatocytes and elicits robust RNAi-mediated gene silencing. Journal of the American Chemical Society, 136(49), pp. 16958-16961.
Soutschek, J., Akinc, A., Bramlage, B., Charisse, K., Constien, R., Donoghue, M., Elbashir, S., Geick, A., Hadwiger, P., Harborth, J. and John, M., 2004. Therapeutic silencing of an endogenous gene by systemic administration of modified siRNAs. Nature, 432(7014), p. 173.

1. A nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human apolipoprotein B protein and wherein said single stranded DNA molecule comprises a nucleotide sequence that is adapted over at least part of its length to anneal by complementary base pairing to a part of said single stranded DNA to form a double stranded DNA structure.

2. A nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human apolipoprotein B protein, or polymorphic sequence variant thereof that varies by one, two or three nucleotides, and wherein said single stranded DNA molecule comprises a nucleotide sequence that is adapted over at least part of its length to anneal by complementary base pairing to a part of said single stranded DNA to form a double stranded DNA structure.

3. The nucleic acid molecule according to claim 1, wherein the 5′ end of said single stranded DNA molecule is covalently linked to the 3′ end of the sense strand of the double stranded inhibitory RNA molecule.

4. The nucleic acid molecule according to claim 1, wherein the 5′ end of said single stranded DNA molecule is covalently linked to the 3′ end of the antisense strand of the double stranded inhibitory RNA molecule.

5. The nucleic acid molecule according to claim 1, wherein said single stranded DNA molecule comprises the nucleotide sequence TCACCTCATCCCGCGAAGC (SEQ ID NO: 1).

6. The nucleic acid molecule according to claim 1, wherein said double stranded inhibitory RNA molecule is between 18 and 29 nucleotides in length.

7. The nucleic acid molecule according to claim 1, wherein said double stranded inhibitory RNA molecule comprises a sense nucleotide sequence selected from the group consisting of: SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57.

8. The nucleic acid molecule according to claim 1, wherein said double stranded inhibitory RNA molecule comprises an antisense nucleotide sequence selected from the group consisting of: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 98, 99, 100, 101, 102, 103, 104, 105, 106, 107, 108, 109 and 110.

9. The nucleic acid molecule according to claim 1, wherein said double stranded inhibitory RNA molecule comprises a sense nucleotide sequence selected from the group consisting of: 111, 113, 115, 117, 119, 121, 123 and 125.

10. The nucleic acid molecule according to claim 1, wherein said double stranded inhibitory RNA molecule comprises an antisense nucleotide sequence selected from the group consisting of: 112, 114, 116, 118, 120, 122, 124 and 126.

11. The nucleic acid molecule according to claim 1, wherein double stranded inhibitory RNA molecule comprises a sense nucleotide sequence selected from the group consisting of: SEQ ID NO: 7, SEQ ID NO: 36, SEQ ID NO: 111, SEQ ID NO: 113, SEQ ID NO: 115 and SEQ ID NO: 119.

12. The nucleic acid molecule according to claim 1, wherein said double stranded inhibitory RNA molecule comprises an antisense nucleotide sequence selected from the group consisting of: SEQ ID NO: 60, SEQ ID NO: 72, SEQ ID NO: 89, SEQ ID NO: 100, SEQ ID NO: 108, SEQ ID NO: 114 and SEQ ID NO: 118.

13. The nucleic acid molecule according to claim 1, wherein:
said double stranded inhibitory RNA molecule comprises a sense nucleotide sequence set forth in SEQ ID NO: 7 and an antisense nucleotide sequence set forth in SEQ ID NO:60;
said double stranded inhibitory RNA molecule comprises a sense nucleotide sequence set forth in SEQ ID NO: 111 and an antisense nucleotide sequence set forth in SEQ ID NO:112;
said double stranded inhibitory RNA molecule comprises a sense nucleotide sequence set forth in SEQ ID NO: 117 and an antisense nucleotide sequence set forth in SEQ ID NO:118;
said double stranded inhibitory RNA molecule comprises a sense nucleotide sequence set forth in SEQ ID NO: 55 and an antisense nucleotide sequence set forth in SEQ ID NO:108;
said double stranded inhibitory RNA molecule comprises a sense nucleotide sequence set forth in SEQ ID NO: 47 and an antisense nucleotide sequence set forth in SEQ ID NO:100;
said double stranded inhibitory RNA molecule comprises a sense nucleotide sequence set forth in SEQ ID NO: 36 and an antisense nucleotide sequence set forth in SEQ ID NO:89;
said double stranded inhibitory RNA molecule comprises a sense nucleotide sequence set forth in SEQ ID NO: 19 and an antisense nucleotide sequence set forth in SEQ ID NO:72;
said double stranded inhibitory RNA molecule comprises a sense nucleotide sequence set forth in SEQ ID NO: 115 and an antisense nucleotide sequence set forth in SEQ ID NO:116;
said double stranded inhibitory RNA molecule comprises a sense nucleotide sequence set forth in SEQ ID NO: 113 and an antisense nucleotide sequence set forth in SEQ ID NO:114;
said double stranded inhibitory RNA molecule comprises a sense nucleotide sequence set forth in SEQ ID NO: 119 and an antisense nucleotide sequence set forth in SEQ ID NO:120; or
said double stranded inhibitory RNA molecule comprises a sense nucleotide sequence set forth in SEQ ID NO: 113 and an antisense nucleotide sequence set forth in SEQ ID NO:114.

14-23. (canceled)

24. The nucleic acid molecule according to claim 1, wherein said nucleic acid molecule is covalently linked to N-acetylgalactosamine.

25. A pharmaceutical composition comprising at least one nucleic acid molecule according to claim 1.

26. A method of treating a subject who has or is predisposed to hypercholesterolemia, comprising administering an effective dose of the pharmaceutical composition according to claim 25, thereby treating or preventing hypercholesterolemia.

27. The method according to claim 26, further comprising treating or preventing a disease associated with hypercholesterolemia.

28. The method according to claim 27, wherein said disease associated with hypercholesterolemia is selected from the group consisting of: familial hypercholesterolemia, stroke prevention, hyperlipidaemia, cardiovascular disease, atherosclerosis, coronary heart disease, aortic stenosis, cerebrovascular disease, peripheral arterial disease, hypertension, metabolic syndrome, type II diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

29. A pharmaceutical composition comprising at least one nucleic acid molecule according to claim 2.

30. A method of treating a subject who has or is predisposed to hypercholesterolemia, comprising administering an effective dose of the pharmaceutical composition according to claim 30, thereby treating or preventing hypercholesterolemia.
